(19) United States
(12) Patent Application Publication
(10) Pub. No.: US 2025/0181871 A1
(43) Pub. Date: Jun. 5, 2025

(54) MULTIMODAL APPARATUS, SYSTEM, AND METHODS OF INSTALLATION AND USE FOR TRACKING OF AN OBJECT

(71) Applicant: FREENAV, INC., St. Paul, MN (US)

(72) Inventors: Brian L. Bechard, St. Paul, MN (US); Mina S. Fahim, New Brighton, MN (US); Greg A. Miller, Findlay, OH (US)

(21) Appl. No.: 18/968,468

(22) Filed: Dec. 4, 2024

Related U.S. Application Data
(60) Provisional application No. 63/605,718, filed on Dec. 4, 2023.

(51) Int. Cl. G06K 19/06

(52) U.S. Cl. CPC G06K 19/06159; G06K 19/06037; G06K 19/0614

(57) ABSTRACT

A multimodal apparatus includes a main body formed from a ferromagnetic material configured for use as both an electromagnetic (EM) tracked fiducial and an optical tracked fiducial with respect to an object to be tracked. The ferromagnetic material enables enhanced versatility through optical tracked fiducial features including QR code patterns, reflective surfaces, surface relief patterns, and non-repeating patterns that provide both visual identification and electromagnetic tracking capabilities. The apparatus allows switching between electromagnetic and optical tracking based on conditions or using both simultaneously to validate accuracy. The multimodal approach provides enhanced tracking capabilities for objects including patients, medical instruments, surgical implants, stents, tracking frames, supply chain components, robotic localization components, motion capture markers, and inventory tracking components. The ferromagnetic material can be configured to produce unique electromagnetic signatures while also incorporating wavelength-specific coatings for optical detection, enabling multimodal tracking without requiring additional dedicated tracking sensors.

### CROSS-REFERENCE TO RELATED APPLICATIONS

This application claims the benefit of U.S. Provisional Application No. 63/605,718, filed on Dec. 4, 2023. The entire disclosure of the above application is hereby incorporated herein by reference.

### FIELD

The present technology relates to sensing systems and methods for determining the location and orientation of an object during procedures including medical, surgical, industrial, and supply chain applications, and, more particularly, to multimodal tracking systems and methods that combine electromagnetic and with other tracking capabilities for enhanced object identification and tracking.

### INTRODUCTION

This section provides background information related to the present disclosure which is not necessarily prior art.

Various systems and methods are known in the art for tracking the location of medical instruments such as catheters, endoscopic capsules, and the like during medical procedures, in order to ascertain their location and orientation during the procedures. Real-time location relative to other structures in the operating arena, for example by overlaying location data onto pre-operative scan data is often beneficial to help guide procedures.

Commercial electromagnetic tracking methods typically either use a permanent magnet or a search coil within the patient. In the first case, the permanent magnet is located by an array of magnetic field sensors, external to the patient. The magnitude and angle of the magnetic field is used to infer the position of the permanent magnet. Such systems are limited by a requirement for a permanent magnet of several cubic millimeters in volume in order to produce a measurable signal, susceptibility to external magnetic fields (such as the earth's field), and inability to track multiple targets.

In the second case, external transmitter coils produce time-varying (AC) magnetic fields and an inductive pick-up coil (search coil) is located within the patient, in which a voltage is induced. Location is inferred by the known properties of the transmitted fields. Such systems also require an internal coil several cubic millimeters in volume, as well as requiring voltages (or data) to be retrieved from the search coil in order for real-time location to be possible. Operation frequency (and hence, signal strength) is limited by the need to avoid distortion in the transmitted fields.

Prior systems are generally hindered by inaccuracies that are caused by the presence of conductive and magnetic materials within the tracking environment. For example, in catheter tracking it is sometimes desirable to place the magnetic field generator of an electromagnetic tracking system on a metal operating room table. This situation also occurs with a 3-dimensional digitizer that is used on a metal desktop.

Known tracking approaches that rely on dedicated electromagnetic tags and sensors also present certain limitations and challenges. The manufacturing and integration of such specialized electromagnetic tracking components is expensive, with complicated manufacturing processes that can present supply chain issues. Modifying existing medical devices and instruments to incorporate dedicated electromagnetic tracking sensors requires significant retooling of manufacturing lines and validation of new processes, increasing costs and potentially compromising the original device functionality.

Current electromagnetic approaches often require cables to connect tracking sensors to external hardware and additional equipment to produce electromagnetic fields, further complicating device design and manufacturing. These limitations have created barriers to widespread adoption of electromagnetic tracking capabilities in medical devices, as manufacturers must choose between expensive redesigns of existing products or foregoing tracking functionality entirely.

One example of a sensing system and method for determining the location and orientation of an object with an EM tag is described in U.S. Patent Application Ser. No. US20200060578 to Pooley, filed on Sep. 18, 2017, and titled “Sensing System and Method,” the entire disclosure of which is hereby incorporated herein by reference. Pooley describes a sensing system involving selection coils and interrogation coils. The selection coils are arranged to generate a spatially-varying DC magnetic field from which the location of the tag can be determined in use. At least some of the interrogation coils are arranged to generate one or more AC magnetic fields and at least some of the interrogation coils are arranged to receive harmonics, intermodulation products or time dependent variations of the AC magnetic fields, from which the orientation of an EM tag is determined in use.

Optical tags, such as reflective surfaces, barcodes, or QR codes, are also known and rely on visual scanning by cameras to retrieve information. These tags are limited in their ability to withstand harsh environments and may become inaccurate or illegible if damaged or dirty. Additionally, optical tracking systems require line-of-sight with a camera mounted a short distance from optical markers, which can be impractical in many medical scenarios.

There is a continuing need for a tracking system and method that overcomes the limitations of both electromagnetic and optical tracking methods used individually. Desirably, such a tracking system and method would address the problems of electromagnetic tracking systems including: decreased accuracy caused by magnetic field distortion, the need for cables to connect tracking sensors, additional equipment required to produce electromagnetic fields, high costs of sensors, complicated manufacturing, supply chain issues, size constraints limiting use with smaller instruments, while also addressing optical-only tracking limitations such as environmental durability, damage susceptibility, and line-of-sight requirements.

### SUMMARY

In concordance with the instant disclosure, a tracking system and method that overcomes the limitations of both electromagnetic and optical tracking methods used individually, and which address the problems of electromagnetic tracking systems alone including decreased accuracy caused by magnetic field distortion, the need for cables to connect tracking sensors, additional equipment required to produce electromagnetic fields, high costs of sensors, complicated manufacturing, supply chain issues, size constraints limiting use with smaller instruments, while also addressing optical-only tracking limitations such as environmental durability, damage susceptibility, and line-of-sight requirements, has surprisingly been discovered.

The present technology includes articles of manufacture, systems, and processes that relate to sensing systems and methods for determining the location and orientation of an object during a procedure such as, but not limited to, medical and surgical procedures, simulations, research, industrial applications, and supply chains and, more particularly, the location and orientation of patient movement during a procedure, and medical devices such as medical instruments, catheters, and implants.

In one embodiment, a multimodal apparatus includes a main body formed from a ferromagnetic material configured for use as both an electromagnetic (EM) tracked fiducial purpose and an optical tracked fiducial purpose with respect to an object to be tracked. The ferromagnetic material enables enhanced versatility through optical tracked fiducial features including QR code patterns that provide both visual identification and electromagnetic tracking capabilities, reflective outer surfaces that enable both camera tracking and electromagnetic field detection, surface relief patterns with depth dimensions that create both optically scannable identifiers and unique electromagnetic signatures, and non-repeating patterns that provide both visual reference points for optical tracking and electromagnetic tracking capabilities.

In another embodiment, a system for multimodal tracking of an object includes a multimodal apparatus having a main body formed from a ferromagnetic material configured for use as both an electromagnetic (EM) tracked fiducial and an optical tracked fiducial, an object to be tracked having an object body on which the multimodal apparatus is disposed, and a sensing system including both an electromagnetic sensing device configured to track the object by electromagnetic means and an optical sensing device configured to identify the object by optical means. The system enables comprehensive tracking through multiple modalities with enhanced reliability and accuracy.

In a further embodiment, a method for installation includes steps of providing an object to be tracked, providing a multimodal apparatus having a main body formed from a ferromagnetic material configured for use as both an electromagnetic (EM) tracked fiducial and an optical tracked fiducial, and installing the multimodal apparatus on the object to be tracked. The multimodal apparatus is configured for both tracking of location and orientation using electromagnetic sensing of the ferromagnetic material and identifying of the object using optical sensing of the optical tracked fiducial. The installation can involve either co-forming the multimodal apparatus with the object during manufacture or attaching it using mechanical fasteners or adhesives.

In yet another embodiment, a method for tracking includes steps of providing an object to be tracked and a multimodal apparatus having a main body formed from a ferromagnetic material configured for use as both an electromagnetic (EM) tracked fiducial and an optical tracked fiducial, tracking location and orientation using electromagnetic sensing of the ferromagnetic material, and identifying the object using optical sensing of the optical tracked fiducial. The method enables switching between electromagnetic and optical tracking based on tracking conditions or using both tracking modes simultaneously for validation purposes.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIGS. 1A to 1E are block diagrams illustrating a multimodal apparatus, according to various embodiments of the present disclosure.

FIG. 2 is a block diagram illustrating a system, according to some embodiments of the present disclosure.

FIG. 3 is a flowchart illustrating a method, according to some embodiments of the present disclosure.

FIG. 4 is a flowchart illustrating a method, according to some embodiments of the present disclosure.

FIG. 5A is a perspective view of an optical tracking frame with reflective spheres having ferromagnetic material disposed in an interior of the reflective spheres, according to one embodiment of the disclosure.

FIG. 5B is a cross-sectional side elevational view of one of the reflective spheres taken at section line X—X in FIG. 5A.

FIG. 6A is a top perspective view of a multimodal apparatus in the form of a thin tag, having a plurality of optical markers disposed on a length of the thin tag with the optical markers provided in a repeating pattern, according to one embodiment of the disclosure.

FIG. 6B is a top perspective view of a multimodal apparatus in the form of a thin tag, having a plurality of optical markers disposed on a length of the thin tag with the optical markers provided in a non-repeating pattern, according to another embodiment of the disclosure.

FIG. 7 is a top perspective view of a single three-dimensional optical marker having a depth dimension, according to one embodiment of the disclosure.

FIG. 8A is a top plan view of a multimodal tag with a three dimensional optical marker that is formed by an additive procedure, the optical marker being a QR code, according to one embodiment of the present disclosure.

FIG. 8B is a cross-sectional side elevational view of a main body of a multimodal tag taken at section line A—A in FIG. 8A.

FIG. 9A is a top plan view of a multimodal tag with a three dimensional optical marker that is formed by a subtractive procedure, the optical marker being a QR code, according to one embodiment of the present disclosure.

FIG. 9B is a cross-sectional side elevational view of a main body of a multimodal tag taken at section line B—B in FIG. 9A.

FIG. 10A is a top plan view of a multimodal tag with a three-dimensional optical marker that is formed by an additive procedure, the optical marker being a bar code with a single depth dimension, according to one embodiment of the present disclosure.

FIG. 10B is a cross-sectional side elevational view of a main body of a multimodal tag taken at section line C—C in FIG. 10A, and showing a single depth dimension associated with the three-dimensional optical marker.

FIG. 11A is a top plan view of a multimodal tag with a three-dimensional optical marker that is formed by an additive procedure, the optical marker being a bar code with a plurality of depth dimensions, according to one embodiment of the present disclosure.

FIG. 11B is a cross-sectional side elevational view of a main body of a multimodal tag taken at section line D—D in FIG. 11A, and showing the plurality of depth dimensions associated with the three-dimensional optical marker.

FIG. 12A is a top plan view of a multimodal tag with a three-dimensional optical marker that is formed by a subtractive procedure, the optical marker being a bar code with a single depth dimension, according to one embodiment of the present disclosure.

FIG. 12B is a cross-sectional side elevational view of a main body of a multimodal tag taken at section line E—E in FIG. 12A, and showing a single depth dimension associated with the three-dimensional optical marker.

FIG. 13A is a top plan view of a multimodal tag with a three-dimensional optical marker that is formed by a subtractive procedure, the optical marker being a bar code with a plurality of depth dimensions, according to one embodiment of the present disclosure.

FIG. 13B is a cross-sectional side elevational view of a main body of a multimodal tag taken at section line F—F in FIG. 13A, and showing a plurality of depth dimensions associated with the three-dimensional optical marker.

FIG. 14A is a top plan view of a multimodal tag with a three-dimensional optical marker that is formed by an additive procedure, the optical marker being a bar code with a plurality of width dimensions between individual bars of the bar code, according to one embodiment of the present disclosure.

FIG. 14B is a cross-sectional side elevational view of a main body of a multimodal tag taken at section line G—G in FIG. 14A, and showing a single depth dimension also associated with the three-dimensional optical marker.

FIG. 15A is a top plan view of a multimodal tag with a three-dimensional optical marker that is formed by both an additive procedure and a subtractive procedure, the optical marker including a plurality of shape elements, according to one embodiment of the present disclosure.

FIG. 15B is a cross-sectional side elevational view of a main body of a multimodal tag taken at section line H—H in FIG. 15A, with a portion of the shape elements formed by the additive procedure being shown.

FIG. 15C is a cross-sectional side elevational view of a main body of a multimodal tag taken at section line I—I in FIG. 15A, with a portion of the shape elements formed by the subtractive procedure being shown.

FIG. 16A is a top plan view of a multimodal tag with a three-dimensional optical marker that is formed by an additive procedure, the optical marker including a plurality of first shape elements and a plurality of second shape elements, according to one embodiment of the present disclosure.

FIG. 16B is a cross-sectional side elevational view of a main body of a multimodal tag taken at section line J—J in FIG. 16A, and showing an arrangement of the first shape elements and the second shape elements.

FIG. 17 is side elevational representation of a sensing system shown in use with a multimodal apparatus for tracked location and orientation, the multimodal apparatus including a tracking frame mounted on a medical instrument, according to some embodiments of the present disclosure.

FIG. 18 is a side elevational representation of a sensing system shown in use with a multimodal apparatus for tracked location and orientation, the multimodal apparatus including a multimodal tag disposed on a medical instrument, and a multimodal tag disposed on a patient, according to other embodiment of the present disclosure.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. Regarding methods disclosed, the order of the steps presented is exemplary in nature, and thus, the order of the steps can be different in various embodiments, including where certain steps can be simultaneously performed, unless expressly stated otherwise. “A” and “an” as used herein indicate “at least one” of the item is present; a plurality of such items may be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that may arise from ordinary methods of measuring or using such parameters.

All documents, including patents, patent applications, and scientific literature cited in this detailed description are incorporated herein by reference, unless otherwise expressly indicated. Where any conflict or ambiguity may exist between a document incorporated by reference and this detailed description, the present detailed description controls.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

As referred to herein, all compositional percentages are by weight of the total composition, unless otherwise specified. Disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, 3-9, and so on.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

The present technology improves tracking accuracy by utilizing a multimodal apparatus having a main body formed from a ferromagnetic material configured for use as both an electromagnetic (EM) tracked fiducial and an optical tracked fiducial with respect to an object to be tracked. The technology enables enhanced versatility through optical tracked fiducial features including QR code patterns that provide both visual identification and electromagnetic tracking capabilities, reflective outer surfaces that enable both camera tracking and electromagnetic field detection, surface relief patterns with depth dimensions that create both optically scannable identifiers and unique electromagnetic signatures, and non-repeating patterns that provide both visual reference points for optical tracking and electromagnetic tracking capabilities. The technology allows switching between electromagnetic tracking and optical tracking based on tracking conditions or using both electromagnetic tracking and optical tracking simultaneously to validate tracking accuracy. The ferromagnetic material can be configured to enter inductor-capacitor resonance or magneto-mechanical resonance at specific AC field frequencies to produce unique electromagnetic signatures, while also being formed with wavelength-specific coatings like IR-reflective coatings adapted for detection by infrared cameras to enable both optical tracking via IR reflection and electromagnetic tracking via the ferromagnetic material. This multimodal approach provides enhanced tracking capabilities for objects including patients having the multimodal apparatus attached as fiducial markers, medical instruments, surgical implants, stents, tracking frames having reflective spheres, supply chain tracking components, robotic localization components, motion capture markers for athletic analysis, video game motion tracking components, and warehouse inventory tracking components.

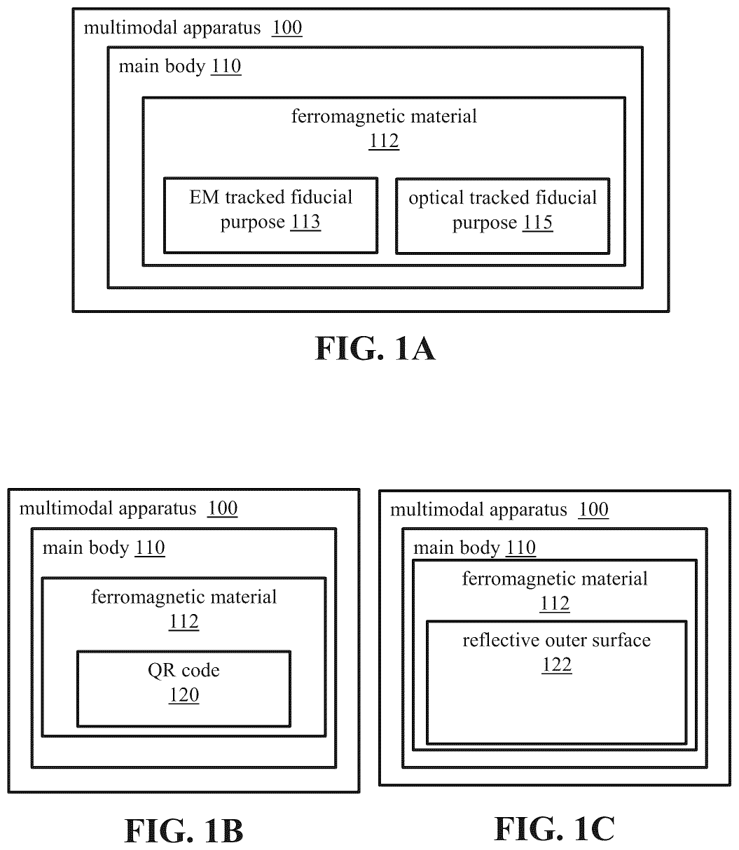

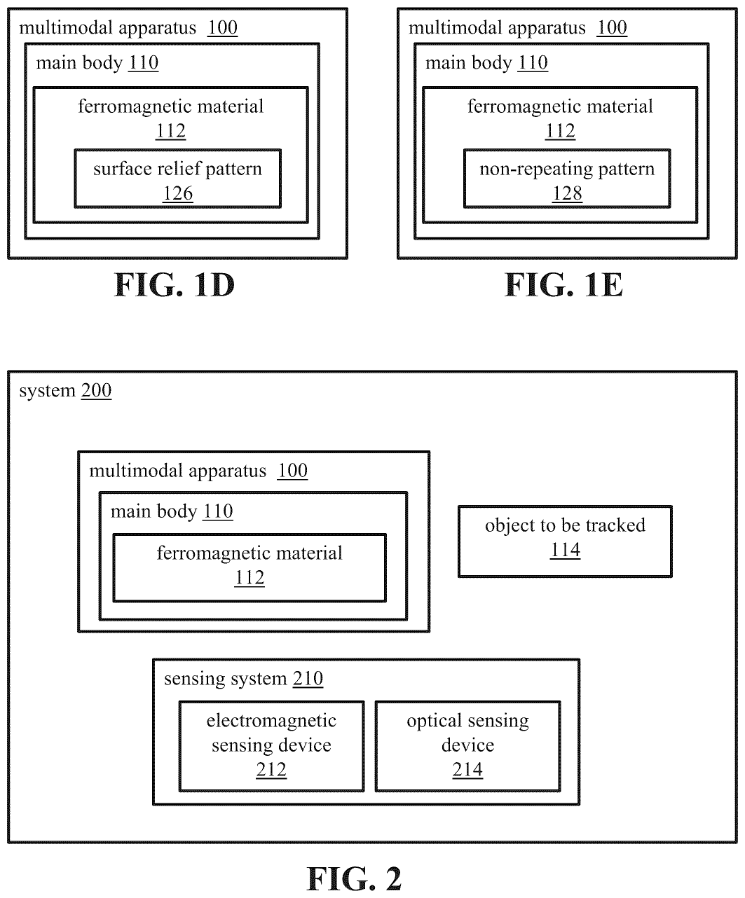

As shown in FIG. 1A, a multimodal apparatus 100 in accordance with the present disclosure can include a main body 110 formed from a ferromagnetic material 112 configured for use for both an electromagnetic (EM) tracked fiducial purpose 113 and an optical tracked fiducial purpose 115 with respect to an object to be tracked 114 (FIG. 2). The ferromagnetic material 112 enables enhanced versatility through multiple optical tracked fiducial features. The main body 110 can be either attached to an object body, or object to be tracked 114 after manufacture or co-formed with the object body, or object to be tracked 114 during manufacture.

The amount of the ferromagnetic material 112 may be selected to provide a desired degree of sensitivity, it being understood that the more material present results in a lower energy of activation required in order for detection by electromagnetic means, for example, as described in U.S. Patent Application Ser. No. US20200060578 to Pooley, filed on Sep. 18, 2017, and titled “Sensing System and Method,” the entire disclosure of which is hereby incorporated herein by reference.

Suitable ferromagnetic materials 112 for use with the system and method of the present disclosure may can include a ferromagnetic material having high magnetic permeability and preferably low coercivity, such as an amorphous or nanocrystalline alloy (e.g. Metglas 2826 MB or the Vacuumschmeltze VITROVAC or VITROPERM materials), for example, as also in Pooley. Other suitable ferromagnetic materials may also be employed by the skilled artisan within the scope of the present disclosure.

The optical tracked fiducial features of the main body 110 can take a variety of different forms, as detailed further herein. In one example, as shown in FIG. 1B, the optical tracked fiducial features of the main body 110 can include a QR code pattern 120 that provides both visual identification and electromagnetic tracking capabilities. The QR code pattern 120 can be provided in either two-dimensional or three-dimensional configurations. For example, the QR code pattern 120 can be formed as a relief in an outer surface of the main body 110.

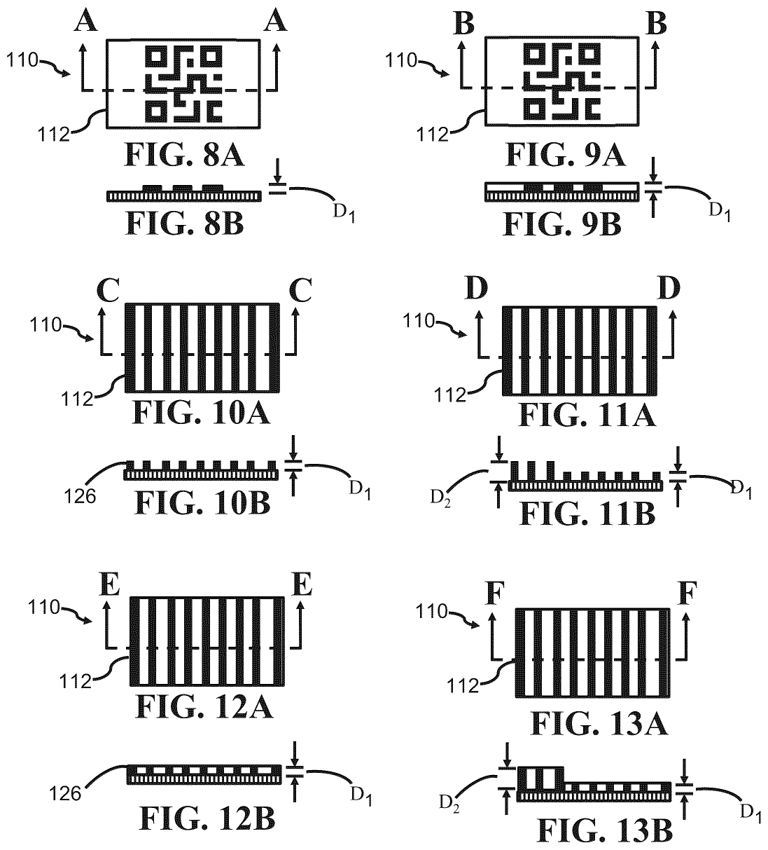

In non-limiting examples, the optical tracked fiducial features of the main body 110 can include QR code patterns 120 that provide both visual identification and electromagnetic tracking capabilities. As illustrated in FIGS. 8A-8B and FIGS. 9A-9B, the ferromagnetic material 112 forming a QR code pattern 120, for example, can be formed in either two-dimensional configurations printed or etched onto the surface of the main body 110, or three-dimensional configurations created through additive or subtractive manufacturing processes.

For additive manufacturing, the QR code pattern 120 can be built up as a relief extending outward from the outer surface of the main body 110, with the depth dimension of the pattern creating both an optically scannable identifier and a unique electromagnetic signature based on the volume and arrangement of the ferromagnetic material.

For subtractive manufacturing, the QR code pattern 120 can be formed by selectively removing material to create recessed features in the outer surface of the main body 110, with the varying depths of the pattern enabling both visual scanning and electromagnetic field detection based on the remaining ferromagnetic material geometry. The three-dimensional QR code configurations provide enhanced functionality by allowing the depth dimension to contribute to both the optical identification capabilities and the electromagnetic tracking signature, while maintaining the ability to be scanned using conventional QR code readers. One of ordinary skill in the art may also select other suitable configurations for forming the two-dimensional or three-dimensional QR code patterns within the scope of the present disclosure, as desired.

As shown in FIG. 1C, the main body 110 can include a reflective outer surface 122 that enables both camera tracking and electromagnetic field detection. The reflective outer surface 122 can be formed by either a reflective surface treatment or a reflective coating, for example. The reflective coating of a reflective sphere 124 can be wavelength-specific, such as an IR-reflective coating adapted for detection by an infrared camera.

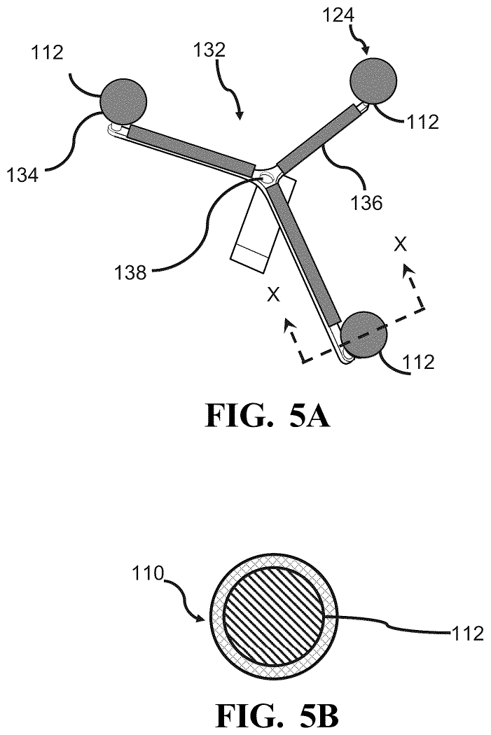

In non-limiting examples, the reflective outer surface 122 can be incorporated into various tracking frame configurations, such as a tracking frame 132 (FIG. 5A) having three arms 136 extending outwardly from a central hub 138, with reflective spheres 124 mounted at the end of each arm 136. The reflective spheres 124 can include ferromagnetic material 112 disposed within an interior core while maintaining a reflective outer surface that enables both optical motion tracking and electromagnetic field detection. The reflective spheres 124 can be configured to serve as reference points relative to one another for precision targeting, calibration, and imaging source registration, with the combination of the reflective outer surface and internal ferromagnetic material allowing simultaneous optical and electromagnetic tracking. The wavelength-specific coatings, such as IR-reflective coatings, can be applied to optimize detection by specific optical tracking systems while preserving the electromagnetic tracking capabilities provided by the internal ferromagnetic cores. One of ordinary skill in the art may also select other suitable configurations for incorporating reflective surfaces with ferromagnetic materials within the scope of the present disclosure, as desired.

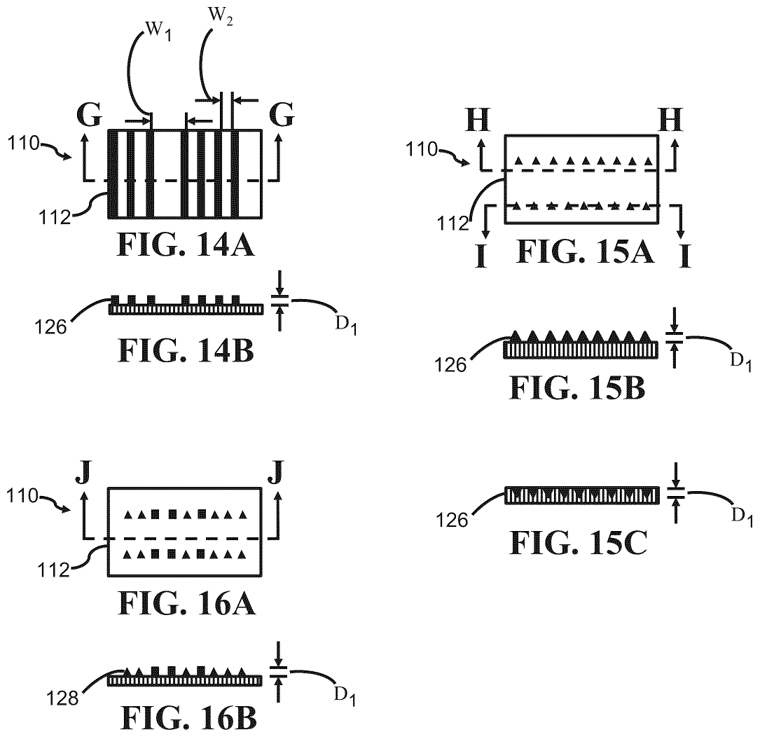

As shown in FIG. 1D, the main body 110 can include a surface relief pattern 126 with a depth dimension that creates both an optically scannable identifier and a unique electromagnetic signature. For example, in addition to the example of the QR code described hereinabove, the surface relief pattern may be in the form of another type of visual code such as a barcode. The surface relief pattern 126 provides both an optically scannable identifier and a unique electromagnetic signature based on a predetermined amount and arrangement of the ferromagnetic material 112 in three dimensions. As shown within FIGS. 8A-16B, the depth dimension D₁ of the surface relief pattern 126 is configured to provide an individual electromagnetic signature unique to an area of the apparatus 100.

The surface relief pattern 126 can be formed by at least one of an additive process and a subtractive process on a substrate of the main body 110, such as shown generally in FIGS. 9A to 16B. For example, the electromagnetic signature may be based on at least one of: a configuration, a volume, a thickness (D₁ and D₂), a width (W₁ and W₂), and a geometry of the ferromagnetic material 112 in the surface relief pattern 126. The surface relief pattern 126 may also comprise a three-dimensional pattern having varying depths that creates a unique harmonic signature when exposed to specific frequency electromagnetic energy.

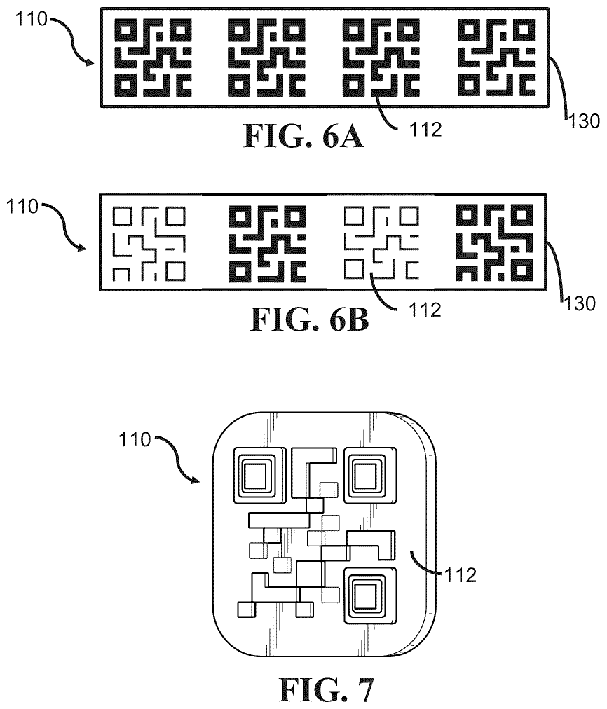

With reference to FIG. 1E, the ferromagnetic material 112 can be distributed in a non-repeating pattern 128 (shown in FIG. 6B and in FIGS. 16A and 16B), distinguishable from a repeating pattern as shown in FIG. 6A, which may provide both visual reference points for optical tracking and electromagnetic tracking capabilities. The non-repeating pattern 128 may have an arrangement of ferromagnetic material 112 that enables production of a plurality of individualized electromagnetic tracking signatures within a range of material content, for example. The non-repeating pattern 128 provides multiple reference points for enhanced tracking accuracy.

In non-limiting examples, the non-repeating pattern 128 can be formed through various manufacturing processes, including additive manufacturing where the ferromagnetic material 112 is selectively deposited to create unique pattern geometries, or subtractive manufacturing where material is selectively removed to create distinctive arrangements. The pattern can incorporate varying thicknesses, densities, and spatial distributions of the ferromagnetic material 112 to generate unique electromagnetic signatures while maintaining optical detectability. The non-repeating nature of the pattern enables individual identification of multiple tags through both their visual appearance and electromagnetic characteristics, with the pattern geometry contributing to both the optical tracking reference points and the electromagnetic field responses. The patterns can be configured to optimize both optical visibility and electromagnetic signature generation through strategic placement and arrangement of the ferromagnetic material 112, allowing simultaneous tracking through both modalities. One of ordinary skill in the art may also select other suitable configurations for creating non-repeating patterns that enable both optical and electromagnetic tracking within the scope of the present disclosure, as desired.

It should be appreciated that the ferromagnetic material 112 may have a high magnetic permeability and low coercivity. The ferromagnetic material 112 can be an amorphous or nanocrystalline alloy selected from: Metglas 2826 MB, VITROVAC, and VITROPERM. The ferromagnetic material 112 is configured to enter an inductor-capacitor resonance or a magneto-mechanical resonance at a frequency of AC magnetic fields to induce a time dependent variation in the AC magnetic fields. One of ordinary skill in the art can also select other suitable ferromagnetic materials 112 for use with the multimodal apparatus 100 and methods 300, 400 within the scope of the present disclosure.

Referring now to FIGS. 6A-6B, 7, and 18, it should be appreciated that in certain embodiments the main body 110 of the multimodal apparatus can be provided in the form of a ribbon tag 130 configured to be disposed on the object body after manufacture. The ribbon tag 130 has specific dimensional characteristics that enhance tracking capabilities. The ribbon tag 130 may have a shape with an aspect ratio greater than 100:1 between short and long edges, for example.

In certain embodiments, the ribbon tag 130 has a thickness of between 0.025 mm and 0.075 mm, and more particularly between 0.00375 mm and 0.00625 mm, and most particularly about 0.005 mm. The ribbon tag 130 has a width of between 0.005 mm and 0.15 mm, and more particularly between 0.075 mm and 0.125 mm, and most particularly about 0.1 mm. The ribbon tag 130 has a length of between 2.5 mm and 7.5 mm, and more particularly between 3.75 mm and 6.25 mm, and most particularly about 5 mm. One of ordinary skill in the art may also select other suitable dimensions for the ribbon tag 130 within the scope of the present disclosure.

It should be appreciated that the ribbon tag 130 can be configured for application in both medical instrumentation and direct patient tracking applications. When applied to instrumentation, for example, as shown in FIG. 18, the ribbon tag 130 can be attached to surgical tools, catheters, endoscopes and other medical devices either during manufacture or as a post-manufacturing addition to enable both optical and electromagnetic tracking during procedures. The tag can be secured through adhesive bonding or mechanical fastening while maintaining its dual tracking capabilities.

For patient applications, as also shown in FIG. 18, the ribbon tag 130 can be attached directly to anatomical landmarks as a fiducial marker to enable registration with pre-operative imaging data and real-time position tracking during procedures. The tag's dimensional characteristics—with thickness between 0.025 mm and 0.075 mm, width between 0.005 mm and 0.15 mm, and length between 2.5 mm and 7.5 mm—allow for minimally intrusive placement on both instruments and patient anatomy while providing sufficient material for generating distinct electromagnetic signatures and maintaining optical visibility. When applied to patients, the tags can serve as reference points for both initial registration and continuous monitoring of patient movement throughout procedures.

The ribbon tag 130 enables seamless tracking across varying procedural conditions, automatically switching between optical and electromagnetic tracking modes based on visibility and access. For both instrumentation and patient tracking applications, the electromagnetic capabilities maintain position monitoring even when optical line-of-sight is blocked by surgical drapes, equipment, or medical personnel, while the optical tracking provides rapid visual verification when available. One of ordinary skill in the art may also select other suitable applications and attachment configurations for the ribbon tag within the scope of the present disclosure, as desired.

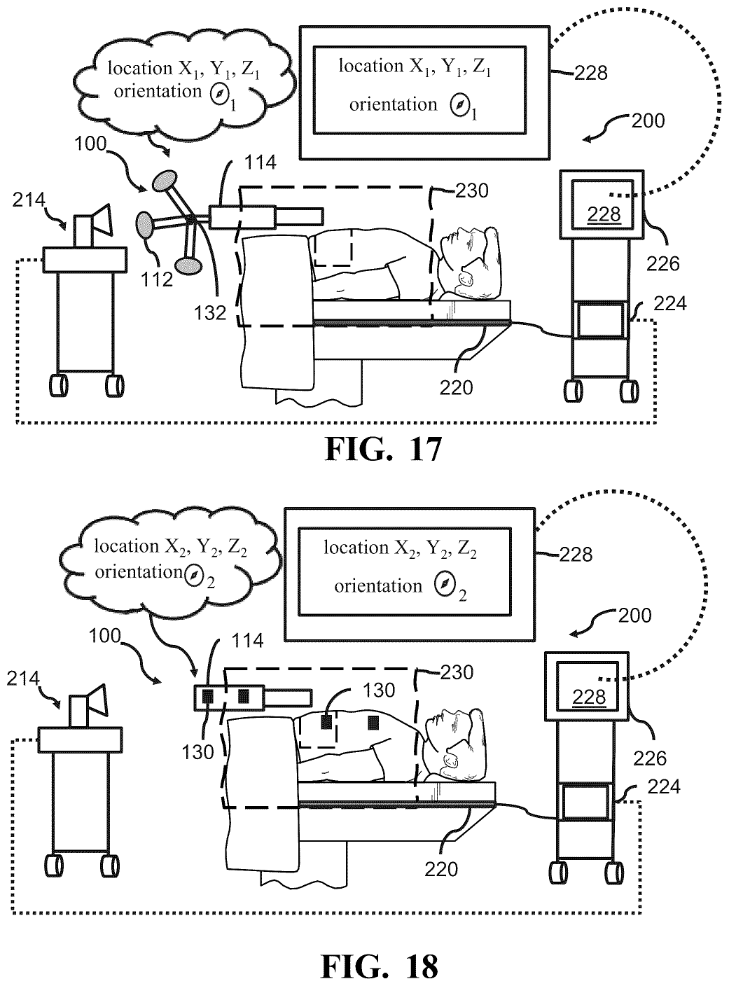

Referring now to FIGS. 5A-5B and 17, in certain embodiments the main body 110 of the multimodal apparatus 100 can include a tracking frame 132 with a plurality of reflective spheres 134. The tracking frame 132 can include three arms 136 extending outwardly from a central hub 138. The plurality of reflective spheres 134 can include three reflective spheres 134, with each of the three reflective spheres 134 mounted to a different one of the three arms 136.

The plurality of reflective spheres 134 are configured to serve as reference points relative to one another for at least one of precision targeting, calibration, and imaging source registration. The reflective spheres 134 enable both optical tracking through their reflective surfaces and electromagnetic tracking through their ferromagnetic construction. The combination of optical and electromagnetic tracking provides enhanced accuracy and reliability.

With reference now to FIG. 2, a system 200 for multimodal tracking can include the multimodal apparatus 100, an object to be tracked 114 having an object body on which the multimodal apparatus 100 is disposed, and a sensing system 210. The sensing system 210 can include an electromagnetic sensing device 212 configured to track the object by electromagnetic means and an optical sensing device 214 configured to identify the object by optical means.

With respect to the multimodal apparatus 100, in general, it should be appreciated that the object to be tracked 114 can include: a patient having the multimodal apparatus 100 attached as a fiducial marker; a medical instrument; a surgical implant; a stent; a tracking frame having reflective spheres; a supply chain tracking component; a robotic localization component; a motion capture marker for athletic analysis; a video game motion tracking component; and a warehouse inventory tracking component.

In a particular example, the multimodal apparatus 100 can be used in medical applications where patient movement tracking is required. The ferromagnetic material 112 enables tracking of patient fiducial markers relative to pre-operative imaging data. The combination of electromagnetic and optical tracking allows compensation for patient movement during procedures.

Without being limited to any particular configuration for sensing the ferromagnetic material 112, the electromagnetic sensing device 212 can include a plurality of coils arranged to generate one or more magnetic fields. At least some of the plurality of coils are arranged to receive harmonics, intermodulation products, or time dependent variations of the magnetic fields. These signals enable determination of the location and orientation of the multimodal apparatus 100.

Likewise, without being limited to any particular configuration for sensing the optical marker, the optical sensing device 214 can include infrared cameras configured to detect IR-reflective coatings on the multimodal apparatus 100. The optical sensing provides redundant tracking capability when electromagnetic tracking is compromised. The multimodal tracking enhance reliability in clinical settings.

With renewed reference to FIGS. 17 and 18, the system 200 may be integrated with an EM field generator 220 for generating an electromagnetic field 230, a position tracking interface 224, and a computing device 226 for controlling an operation of the system 200 through one or more executable instructions stored in a memory of the computing device 226 and displayed on a screen 228.

For example, the EM field generator 220 may be configured for determination of a location and an orientation of the device 100 such as according to the methods described in U.S. Patent Application Ser. No. 20200060578 to Pooley. The position tracking interface 224 may be configured for transmitting the EM field generator 220 and sending real-time location and orientation data of sensors such as the device 100 including the ferromagnetic material 112. As further shown in FIGS. 17 and 18, a location and orientation or poise of the device 100 may thereby be displayed on screen 228 of the system 200 in a manner that is consistent with the actual location and orientation of the device 100 in operation.

For medical instrument tracking applications, the multimodal apparatus 100 can be integrated into catheter braids, stent structures, or surgical tool components. The ferromagnetic material 112 provides both structural support and tracking functionality. This multimodal design eliminates the need for separate tracking components.

In surgical navigation applications, the multimodal apparatus 100 can be incorporated into reference frames and targeting devices. The combination of optical and electromagnetic tracking enables precise alignment with pre-operative imaging. The tracking frame design with reflective spheres 134 provides multiple reference points.

For implant tracking, the multimodal apparatus 100 can be integrated into devices such as stents, orthopedic implants, and therapeutic implants. The ferromagnetic material 112 enables post-implantation location verification. The optical features allow intraoperative positioning guidance.

In robotic surgery applications, the multimodal apparatus 100 can be incorporated into end effectors and surgical tools. The electromagnetic tracking provides real-time position data for robotic control. The optical tracking enables visual verification of tool positions.

For supply chain and inventory tracking, the multimodal apparatus 100 can be applied as labels or integrated into packaging. The QR code patterns provide visual identification and inventory management. The electromagnetic tracking enables automated location tracking.

In motion capture applications, the multimodal apparatus 100 can be incorporated into markers and tracking suits. The reflective surfaces enable traditional optical motion capture. The electromagnetic tracking provides data when optical tracking is occluded.

For gaming and virtual reality applications, the multimodal apparatus 100 can be integrated into controllers and tracking accessories. The combination of optical and electromagnetic tracking enhances motion detection accuracy. The multiple modalities enable consistent tracking in varying lighting conditions.

The system 200 can be configured to automatically switch between tracking modes based on environmental conditions. The electromagnetic tracking can take over when optical line-of-sight is blocked. The optical tracking can provide primary tracking when electromagnetic interference is present.

The system 200 can use both tracking modes simultaneously for enhanced accuracy validation. The optical tracking data can be compared to electromagnetic tracking data. This comparison enables detection of tracking errors or interference.

The installation method 300 can include steps for calibrating and testing both tracking modalities. The optical tracking features are verified using the optical sensing device 214. The electromagnetic tracking is validated using the electromagnetic sensing device 212.

The tracking method 400 can include continuous monitoring of tracking quality from both modalities. The system can automatically select the optimal tracking mode based on signal quality. The multimodal tracking capabilities ensure consistent performance across varying conditions.

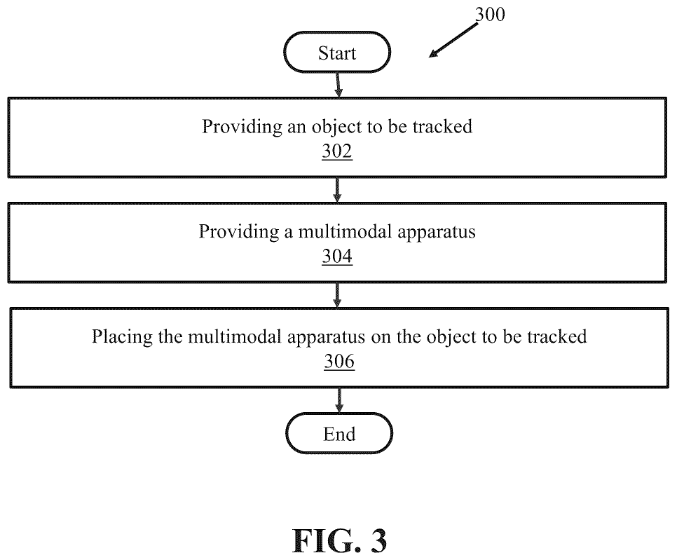

As shown in FIG. 3, a method 300 for installation of the multimodal apparatus 100 described hereinabove can include: a first step 302 of providing an object to be tracked; a second step 304 of providing the multimodal apparatus 100; and a third step 306 of placing the multimodal apparatus 100 on the object to be tracked. The multimodal apparatus 100 is configured for both tracking of location and orientation using electromagnetic sensing and identifying using optical sensing.

It should be appreciated that the third step 306 of placing the multimodal apparatus 100 can take many forms. For example, the third step 306 of placing can include co-forming the multimodal apparatus 100 with the object or attaching the multimodal apparatus 100 to the object with one of a mechanical fastener and an adhesive. The installation method 300 can further include forming optical tracking features such as QR codes, reflective surfaces, and relief patterns. The method 300 ensures proper integration of both electromagnetic and optical tracking capabilities in the singular multimodal apparatus 100. One of ordinary skill in the art can also select other suitable steps for use with the method 300 within the scope of the present disclosure.

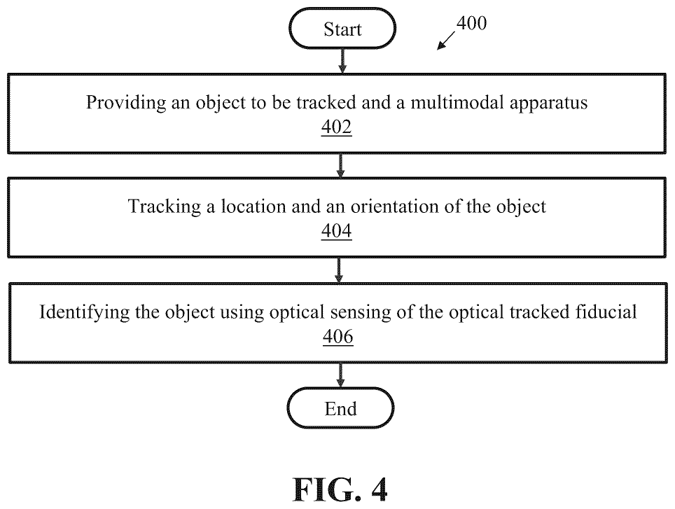

As shown in FIG. 4, a method 400 for tracking the object through use of the multimodal apparatus 100 described hereinabove can include: a fourth step 402 of providing an object to be tracked and the multimodal apparatus 100; a fifth step 404 of tracking location and orientation using electromagnetic sensing; and a sixth step 406 of identifying the object using optical sensing. The method 400 enables comprehensive tracking through multiple modalities.

The method 400 can further include a step of detecting harmonics or intermodulation products of an AC magnetic field generated by the ferromagnetic material 112. The method can include a step of scanning a QR code formed as a relief pattern in the main body 110. The method can include a step detecting reflected light from a reflective surface of the main body 110 using an optical tracking system.

The method 400 can include a step of switching between electromagnetic tracking and optical tracking based on tracking conditions. The method can include a step of using both electromagnetic tracking and optical tracking simultaneously to validate tracking accuracy. This multimodal approach provides enhanced reliability and flexibility in tracking applications. One of ordinary skill in the art can also select other suitable steps for use with the method 400 within the scope of the present disclosure.

Advantageously, the multimodal apparatus and methods of the present disclosure provide a comprehensive solution that addresses the limitations of both electromagnetic and optical tracking methods used individually by combining a ferromagnetic material configured for dual-purpose use as both an EM tracked fiducial and an optical tracked fiducial. The ferromagnetic material enables wireless unpowered tracking through natural harmonic and intermodulation responses while eliminating the need for cables and additional dedicated tracking sensors that increase costs and manufacturing complexity. The optical tracked fiducial features including QR codes, reflective surfaces, and surface relief patterns overcome the environmental durability and line-of-sight limitations of conventional optical-only tags, while the electromagnetic tracking capabilities address accuracy issues caused by magnetic field distortion and equipment constraints that limit traditional EM-only systems. By enabling switching between tracking modes based on conditions or simultaneous use for validation, the multimodal approach provides enhanced reliability and accuracy across varying environments while maintaining original device functionality without requiring modifications to conventional manufacturing processes.

### EXAMPLES

Example embodiments of the present technology are provided with reference to the several figures enclosed herewith. Although the present disclosure is described largely herein and below with respect to the field of medical technology, it should also be understood that applications of the present disclosure are not limited to the medical field and that other non-medical applications are contemplated and considered to be within the scope of the present disclosure.

### Example 1: Surgical Navigation Frame with Multimodal Tracking

A surgical navigation frame incorporates a tracking frame having three arms extending outwardly from a central hub, with reflective spheres mounted at the end of each arm. The reflective spheres enable both optical motion tracking through their reflective outer surfaces and electromagnetic tracking through ferromagnetic cores. The spheres are coated with wavelength-specific IR-reflective coatings adapted for detection by infrared cameras while maintaining their electromagnetic tracking capabilities.

The reflective spheres serve as reference points relative to one another for precision targeting, calibration, and imaging source registration. The system can automatically switch between optical tracking via the IR-reflective coatings and electromagnetic tracking via the ferromagnetic cores based on tracking conditions. When line of sight to the optical tracking cameras is blocked, the system seamlessly transitions to electromagnetic tracking mode.

The system can also use both tracking modes simultaneously to validate accuracy, comparing the optical tracking data to the electromagnetic tracking data to detect any errors or interference. This multimodal redundancy ensures consistent and reliable tracking performance during surgical navigation procedures.

### Example 2: Smart Medical Device with QR Code Tracking

A medical device incorporates a QR code pattern formed as a relief in the ferromagnetic material, enabling both visual identification and electromagnetic tracking capabilities. The three-dimensional QR code pattern creates a unique electromagnetic signature based on its depth dimension while also providing an optically scannable identifier. The pattern can be scanned visually for rapid identification while simultaneously enabling electromagnetic field detection.

The system can track the device using both optical scanning of the QR code and electromagnetic sensing of the ferromagnetic material pattern. The optical tracking provides quick visual verification of device identity and position, while the electromagnetic tracking enables continuous monitoring even when the QR code is not visible. The dual tracking modes enhance reliability in clinical settings.

The combination of optical and electromagnetic tracking allows seamless transitions between tracking modes based on environmental conditions. When optical scanning is impractical due to viewing angle or obstruction, the system maintains tracking through the electromagnetic signature of the QR code pattern. This flexibility ensures consistent device tracking throughout procedures.

### Example 3: Implant with Surface Relief Pattern

An implant features a surface relief pattern with varying depths that creates both an optically scannable identifier and a unique electromagnetic signature. The pattern provides visual reference points for optical tracking while its three-dimensional geometry generates characteristic electromagnetic field responses. The depth variations in the pattern create individualized electromagnetic signatures based on the volume and arrangement of the ferromagnetic material.

The surface relief pattern enables dual-mode tracking during both implantation and follow-up procedures. The optical features allow direct visual tracking during placement, while the electromagnetic signatures enable position verification even after implantation when the pattern is no longer visible. The system can validate tracking accuracy by comparing optical and electromagnetic data.

The multimodal tracking capabilities enhance both acute and chronic monitoring applications. During procedures, the system can switch between optical and electromagnetic tracking based on visibility conditions. Post-implantation, the electromagnetic signatures enable long-term position monitoring while the optical pattern remains available for visual confirmation when accessible.

### Example 4: Motion Capture System with Multimodal Markers

A motion capture system uses markers featuring reflective outer surfaces for optical tracking combined with ferromagnetic cores for electromagnetic tracking. The markers are coated with wavelength-specific reflective materials for enhanced optical detection while maintaining their electromagnetic properties. This multimodal design enables simultaneous tracking through both optical motion capture cameras and electromagnetic field detection.

The system tracks multiple markers concurrently using both modalities, automatically selecting the optimal tracking mode based on signal quality. The optical tracking provides high-frequency position updates when line-of-sight is maintained, while the electromagnetic tracking ensures continuous data collection even when markers are occluded. The system can validate tracking accuracy by comparing data from both modes.

This multimodal approach overcomes traditional motion capture limitations by providing reliable tracking in varying lighting conditions and through occlusions. The system achieves consistent performance in challenging environments by seamlessly switching between or combining optical and electromagnetic tracking modes. The redundant tracking capabilities enhance data quality for applications in gaming, sports analysis, and animation.

### Example 5: Warehouse Inventory Tracking System

An inventory tracking system employs tags with non-repeating patterns that provide both visual reference points and electromagnetic tracking capabilities. The patterns enable optical scanning for rapid identification while generating unique electromagnetic signatures for wireless tracking. The combination allows both visual inventory management and automated position monitoring.

The system can track items using either optical scanning of the patterns or electromagnetic detection of their unique signatures. The optical tracking enables quick visual verification during manual inventory checks, while the electromagnetic tracking provides continuous automated monitoring of item locations. The dual-mode approach enhances inventory management efficiency.

The multimodal tracking capabilities enable flexible operation in varying warehouse conditions. When direct scanning is impractical due to item placement or lighting, the system maintains tracking through electromagnetic signatures. The ability to switch between or combine tracking modes ensures reliable inventory monitoring across different environments and handling scenarios.

### Example 6: Patient Fiducial Markers with Multimodal Tracking

A patient tracking system employs multimodal fiducial markers attached to anatomical landmarks to enable registration with pre-operative imaging data. The fiducial markers incorporate both reflective spheres mounted on a tracking frame for optical tracking and ferromagnetic cores for electromagnetic tracking. The reflective spheres are coated with wavelength-specific IR-reflective materials that enable detection by infrared cameras while the ferromagnetic cores generate unique electromagnetic signatures.

The system can track patient movement using both optical scanning of the reflective spheres and electromagnetic detection of the ferromagnetic cores. During procedures, the optical tracking provides real-time visualization of patient position relative to surgical instruments and imaging equipment, while the electromagnetic tracking maintains continuous monitoring even when optical line-of-sight is blocked by surgical drapes or equipment. The system can automatically switch between tracking modes based on environmental conditions or use both simultaneously to validate tracking accuracy.

The multimodal approach enhances patient tracking reliability by providing redundant position verification methods. When surgical team members or equipment obstruct the optical tracking cameras' view of the reflective spheres, the system seamlessly transitions to electromagnetic tracking mode to maintain continuous patient position monitoring. The multimodal tracking capabilities ensure consistent registration between real-time patient position and pre-operative imaging throughout procedures, enabling precise navigation and targeting even with patient movement.

The fiducial markers can be configured in various arrangements on the patient to establish multiple reference points for enhanced tracking accuracy. The system uses these reference points for both initial registration with pre-operative imaging and continuous monitoring during procedures. The combination of optical and electromagnetic tracking modalities enables the system to maintain accurate patient position data across varying operating room conditions and procedural requirements.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions and methods can be made within the scope of the present technology, with substantially similar results.

What is claimed is:

1. A multimodal apparatus, comprising: a main body formed from a ferromagnetic material configured for use as both an electromagnetic (EM) tracked fiducial purpose and an optical tracked fiducial purpose with respect to an object to be tracked.

2. The multimodal apparatus of claim 1, wherein the optical tracked fiducial purpose includes at least one of:
the ferromagnetic material having a QR code pattern that provides both visual identification and electromagnetic tracking capabilities;
the ferromagnetic material has a reflective outer surface that enables both camera tracking and electromagnetic field detection;
the ferromagnetic material having a surface relief pattern with a depth dimension that creates both an optically scannable identifier and a unique electromagnetic signature; and
the ferromagnetic material being distributed in a non-repeating pattern that provides both visual reference points for optical tracking and electromagnetic tracking capabilities.

3. The multimodal apparatus of claim 2, wherein the QR code pattern is formed as a relief in an outer surface of the main body.

4. The multimodal apparatus of claim 2, wherein the QR code pattern is one of a two-dimensional QR code pattern or a three-dimensional QR code pattern.

5. The multimodal apparatus of claim 2, wherein the reflective outer surface is formed by at least one of a reflective surface treatment and a reflective coating.

6. The multimodal apparatus of claim 5, wherein the reflective coating is a wavelength-specific coating.

7. The multimodal apparatus of claim 6, wherein the wavelength-specific coating is an IR-reflective coating adapted for detection by an infrared camera and applied to at least a portion of the ferromagnetic material to enable both optical tracking via IR reflection and electromagnetic tracking via the ferromagnetic material.

8. The multimodal apparatus of claim 2, wherein the main body includes a tracking frame and a plurality of reflective spheres, the reflective spheres providing the reflective outer surface.

9. The multimodal apparatus of claim 8, wherein the tracking frame includes three arms extending outwardly from a central hub, and the plurality of reflective spheres includes three reflective spheres, and each of the three reflective spheres is mounted to a different one of the three arms.

10. The multimodal apparatus of claim 2, wherein the surface relief pattern provides both an optically scannable identifier and a unique electromagnetic signature based on a predetermined amount and arrangement of the ferromagnetic material in the three dimensions of the pattern.

11. The multimodal apparatus of claim 2, wherein the depth dimension of the surface relief pattern is configured to provide an individual electromagnetic signature unique to an area of the apparatus at which the surface relief pattern is located based on a volume and a geometry of the ferromagnetic material.

12. The multimodal apparatus of claim 2, wherein the surface relief pattern has the non-repeating pattern of ferromagnetic material that enables production of a plurality of individualized electromagnetic tracking signatures within a range of material content.

13. The multimodal apparatus of claim 2 wherein the surface relief pattern is formed by one of an additive process and a subtractive process on a substrate of the main body.

14. The multimodal apparatus of claim 1, wherein the main body is provided in the form of a ribbon tag configured to be disposed on the object body after manufacture.

15. The multimodal apparatus of claim 14, wherein the ribbon tag has a shape with an aspect ratio greater than 100:1 between short and long edges.

16. The multimodal apparatus of claim 1, wherein the main body is co-formed with the object during manufacture.

17. A method for installation, the method comprising steps of:
providing an object to be tracked;
providing a multimodal apparatus having a main body formed from a ferromagnetic material configured for use as both an electromagnetic (EM) tracked fiducial and an optical tracked fiducial with respect to the object to be tracked; and
placing the multimodal apparatus on the object to be tracked, wherein the multimodal apparatus is configured for both a tracking of a location and orientation of the object using electromagnetic sensing of the ferromagnetic material, and an identifying of the object using optical sensing of the optical tracked fiducial.

18. The method of claim 17, wherein the step of installing the multimodal apparatus on the object includes one of: a co-forming of the multimodal apparatus with the object to be tracked; and attaching the multimodal apparatus to the object with one of a mechanical fastener and an adhesive.

19. A method for tracking, the method comprising steps of:
providing an object to be tracked and a multimodal apparatus having a main body formed from a ferromagnetic material configured for use as both an electromagnetic (EM) tracked fiducial and an optical tracked fiducial with respect to the object to be tracked;
tracking a location and an orientation of the object using electromagnetic sensing of the ferromagnetic material; and
identifying the object using optical sensing of the optical tracked fiducial.

20. The method of claim 19, further comprising one of: a step of switching between electromagnetic tracking and optical tracking based on tracking conditions; and a step of using both electromagnetic tracking and optical tracking simultaneously to validate tracking accuracy.
